SEQUENCE LISTING (2 sequences)

SEQ ID NO: 1 - protein, 349 aa, HOMO SAPIENS
MNSTLDGNQSSHPFCLLAFGYLETVNFCLLEVLIIVFLTVLIISGNIIVIFVFHCAPLLNHHTTSYFIQTMAYADLFVGVSCVVPSLSLLHHPLPVEESLTCQIFGFVVSVLKSVSMASLACISIDRYIAITKPLTYNTLVTPWRLRLCIFLIWLYSTLVFLPSFFHWGKPGYHGDVFQWCAESWHTDSYFTLFIVMMLYAPAALIVCFTYFNIFRICQQHTKDISERQARFSSQSGETGEVQACPDKRYAMVLFRITSVFYILWLPYIIYFLLESSTGHSNRFASFLTTWLAISNSFCNCVIYSLSNSVFQRGLKRLSGAMCTSCASQTTANDPYTVRSKGPLNGCHI

SEQ ID NO: 2 - DNA, 1050 bp, HOMO SAPIENS
atgaactccaccttggatggtaatcagagcagccaccctttttgcctcttggcatttggctatttggaaactgtcaatttttgccttttggaagtattgattattgtctttctaactgtattgattatttctggcaacatcattgtgatttttgtatttcactgtgcacctttgttgaaccatcacactacaagttattttatccagactatggcatatgctgacctttttgttggggtgagctgcgtggtcccttctttatcactcctccatcacccccttccagtagaggagtccttgacttgccagatatttggttttgtagtatcagttctgaagagcgtctccatggcttctctggcctgtatcagcattgatagatacattgccattactaaacctttaacctataatactctggttacaccctggagactacgcctgtgtattttcctgatttggctatactcgaccctggtcttcctgccttcctttttccactggggcaaacctggatatcatggagatgtgtttcagtggtgtgcggagtcctggcacaccgactcctacttcaccctgttcatcgtgatgatgttatatgccccagcagcccttattgtctgcttcacctatttcaacatcttccgcatctgccaacagcacacaaaggatatcagcgaaaggcaagcccgcttcagcagccagagtggggagactggggaagtgcaggcctgtcctgataagcgctatgccatggtcctgtttcgaatcactagtgtattttacatcctctggttgccatatatcatctacttcttgttggaaagctccactggccacagcaaccgcttcgcatccttcttgaccacctggcttgctattagtaacagtttctgcaactgtgtaatttatagtctctccaacagtgtattccaaagaggactaaagcgcctctcaggggctatgtgtacttcttgtgcaagtcagactacagccaacgacccttacacagttagaagcaaaggccctcttaatggatgtcatatctga

(19) United States
(12) Patent Application Publication
(10) Pub. No.: US 2002/0106630 A1
(43) Pub. Date: Aug. 8, 2002

(54) Method of identifying HIBEF51 receptor inhibitors

(76) Inventors: Janet Elaine Park, Harrogate (GB); Jon Chambers, Haslingfield (GB)

Correspondence Address: Ratner & Prestia, P.O. Box 980, Valley Forge, PA, 19482-0980, US

(21) Appl. No.: 09/916,051

(22) Filed: Jul. 26, 2001

Related U.S. Application Data
(63) Continuation of application No. 09/201,288, filed on Nov. 30, 1998, now abandoned.

(30) Foreign Application Priority Data
Dec. 1, 1997 (GB) 9725462.7

(51) Int. Cl. C12Q 1/00; C12Q 1/44; A61K 31/00

(52) U.S. Cl. 435/4; 435/19; 514/1

(57) ABSTRACT

A method for identifying compounds which inhibit the HIBEF51 receptor activity.

This invention relates to the use of a human G-protein coupled receptor as a screening tool to identify inhibitors of the adenylyl cyclase activity resulting from expression of the receptor.

It is well established that many medically significant biological processes are mediated by proteins participating in signal transduction pathways that involve G-proteins and/or second messengers, e.g., cAMP (Lefkowitz, Nature, 1991, 351:353-354). Herein these proteins are referred to as proteins participating in pathways with G-proteins or PPG proteins. Some examples of these proteins include the GPC receptors, such as those for adrenergic agents and dopamine (Kobilka, B. K., et al., Proc. Natl Acad. Sci., USA, 1987, 84:46-50; Kobilka, B. K., et al., Science, 1987, 238:650-656; Bunzow, J. R., et al., Nature, 1988, 336:783-787), G-proteins themselves, effector proteins, e.g., phospholipase C, adenylyl cyclase, and phosphodiesterase, and actuator proteins, e.g., protein kinase A and protein kinase C (Simon, M. I., et al., Science, 1991, 252:802-8).

For example, in one form of signal transduction, the effect of hormone binding is activation of the enzyme, adenylyl cyclase, inside the cell. Enzyme activation by hormones is dependent on the presence of the nucleotide GTP. GTP also influences hormone binding. A G-protein connects the hormone receptor to adenylyl cyclase. G-protein was shown to exchange GTP for bound GDP when activated by a hormone receptor. The GTP-carrying form then binds to activated adenylyl cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane a-helices connected by extracellular or cytoplasmic loops. G-protein coupled receptors include a wide range of biologically active receptors, such as hormone, viral, growth factor and neuroreceptors.

G-protein coupled receptors (otherwise known as 7TM receptors) have been characterized as including these seven conserved hydrophobic stretches of about 20 to 30 amino acids, connecting at least eight divergent hydrophilic loops. The G-protein family of coupled receptors includes dopamine receptors which bind to neuroleptic drugs used for treating psychotic and neurological disorders. Other examples of members of this family include, but are not limited to, calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant, and cytomegalovirus receptors.

Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or famesylation) of cysteine residues can influence signal transduction of some G-protein coupled receptors. Most G-protein coupled receptors contain potential phosphorylation sites within the third cytoplasmic loop and/or the carboxy terminus. For several G-protein coupled receptors, such as the b-adrenoreceptor, phosphorylation by protein kinase A and/or specific receptor kinases mediates receptor desensitization.

For some receptors, the ligand binding sites of G-protein coupled receptors are believed to comprise hydrophilic sockets formed by several G-protein coupled receptor transmembrane domains, said socket being surrounded by hydrophobic residues of the G-protein coupled receptors. The hydrophilic side of each G-protein coupled receptor transmembrane helix is postulated to face inward and form polar ligand binding site. TM3 has been implicated in several G-protein coupled receptors as having a ligand binding site, such as the TM3 aspartate residue. TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 1989, 10:317-331) Different G-protein a-subunits preferentially stimulate particular effectors to modulate various biological functions in a cell. Phosphorylation of cytoplasmic residues of G-protein coupled receptors have been identified as an important mechanism for the regulation of G-protein coupling of some G-protein coupled receptors. G-protein coupled receptors are found in numerous sites within a mammalian host.

Over the past 15 years, nearly 350 therapeutic agents targeting 7 transmembrane (7 TM) receptors have been successfully introduced onto the market.

Therapeutic agents targeting 7-TM receptors can play a role in preventing, ameliorating or correcting dysfunctions or diseases, including, but not limited to, infections such as bacterial, fungal, protozoan and viral infections, particularly infections caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, depression, migraine, vomiting, stroke, schizophrenia, manic depression, delirium, dementia, severe mental retardation and dyskinesias, such as Huntington's disease or Gilles dela Tourett's syndrome.

A human G-protein coupled receptor known as HIBEF51 is disclosed in WO96/39441 and O'Dowd et al., Gene 187 (1997) 75-81. No natural ligand for the receptor is disclosed. The receptor gene is localised to chromosome 9q33.3-34. Disease states which are localised to this area include hypomelanosis, lethal congenital contracture syndrome, nail patella syndrome, tuberous sclerosis, dystonia, abelson murine leukemia, retinitis pigmentosa-deafness syndrome, stomatocytosis, deficiency of complement component 5 and citrullinemia. Diseases which associate with the mouse syntenous region include muscular dystrophy with myositis, rachiterata, pallid, mahogany, wellhaarig and coloboria. The polypeptide sequence of HIBEF51 is given as SEQ ID NO: 1 and a polynucleotide sequence encoding HIBEF51 as SEQ ID NO: 2.

It has now been found that HIBEF51 is a constitutively active receptor and is coupled to adenylyl cyclase.

Inhibitors of the constitutive activity of the receptor (inverse agonists) are useful in the prevention and/or treatment of infections such as bacterial, fungal, protozoan and viral infections, particularly infections caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, depression, migraine, vomiting, stroke, schizophrenia, manic depression, delirium, dementia, severe mental retardation, dyskinesias, such as Huntington's disease or Gilles dela Tourett's syndrome, hypomelanosis, lethal congenital contracture syndrome, nail patella syndrome, tuberous sclerosis, dystonia, abelson murine leukemia, retinitis pigmentosa-deafness syndrome, stomatocytosis, deficiency of complement component 5 and citrullinemia, hereinafter ‘the Diseases’.

According to the invention there is provided a method for identifying compounds which inhibit the HIBEF51 receptor activity which comprises:

(a) contacting a candidate compound with cells which express the polypeptide of SEQ ID NO: 1, or with cell membranes prepared from said cells, in the absence of any ligand; and

(b) observing the inhibition of adenylyl cyclase activity.

Preferably the observation (b) is of the reduction of the production and/or accumulation of cAMP.

Suitable cell lines are well known in the art and include, for instance, eukaryotic cells such as mammalian, amphibian, yeast and drosophila. Preferably the cell line is mammalian, such as HEK293, COS or CHO or amphibian such as frog melanophore. Receptor expression may be transient or stable. Preferably, the expression is stable.

Preferably, a cell line is transfected with an expression vector comprising a nucleic acid sequence encoding the HIBEF51 receptor, and the cell line then cultured in a culture medium, such that the receptor domain is stably expressed within the outer membrane of the cell.

In one aspect the method of the present invention may employ the yeast based technology as described in U.S. Pat. No. 5,482,835.

Inhibitors of the receptor activity may be identified from a variety of sources, for instance, from cells, cell-free preparations, chemical libraries and natural product mixtures.

Compounds identified using the screen will be of use in therapy. Accordingly, in a further aspect, the present invention provides a compound identified as an inhibitor of the HIBEF51 receptor activity for use in therapy.

Compounds thus identified may be used for treating abnormal conditions related to an excess of HIBEF51 receptor activity, in particular any of the Diseases mentioned above.

Accordingly, in a further aspect, this invention provides a method of treating an abnormal condition related to an excess of HIBEF51 receptor activity which comprises administering to a patient in need thereof an inhibitor as hereinbefore described in an amount effective to inhibit receptor activity, and thereby alleviating the abnormal condition.

Compounds for use in such methods of treatment will normally provided in pharmaceutical compositions. Accordingly, in a further aspect, the present invention provides a pharmaceutical composition comprising a compound identified as an inhibitor of the HIBEF51 receptor and a pharmaceutically acceptable excipient or carrier.

Compounds which are active when given orally can be formulated as liquids, for example syrups, suspensions or emulsions, tablets, capsules and, lozenges.

A liquid formulation will generally consist of a suspension or solution of the compound or pharmaceutically acceptable salt in a suitable liquid carrier(s) for example, ethanol, glycerine, non-aqueous solvent, for example polyethylene glycol, oils, or water with a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

A typical suppository formulation comprises a compound of formula (I) or a pharmaceutically acceptable salt thereof which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule.

Each dosage unit for oral administration contains preferably from 1 to 250 mg (and for parenteral administration contains preferably from 0.1 to 25 mg) of an inhibitor of the invention.

The daily dosage regimen for an adult patient may be, for example, an oral dose of between 1 mg and 500 mg, preferably between 1 mg and 250 mg, or an intravenous, subcutaneous, or intramuscular dose of between 0.1 mg and 100 mg, preferably between 0.1 mg and 25 mg, of the inhibitor, the compound being administered 1 to 4 times per day. Suitably the compounds will be administered for a period of continuous therapy.

The invention is further described in the following examples which are intended to illustrate the invention without limiting its scope.

All publications, including but not limited to patents and patent applications, cited in this specification are herein incorporated by reference as if each individual publication were specifically and individually indicated to be incorporated by reference herein as though fully set forth.

SEQ ID NO 1 (Polypeptide)

MNSTLDGNQSSHPFCLLAFGYLETVNFCLLEVLIIVFLTVLIISGNIIVIFVFHCAPLL
NHHTTSYFIQTMAYADLFVGVSCVVPSLSLLHHPLPVEESLTCQIFGFVVSVLKSV
SMASLACISIDRYIAITKPLTYNTLVTPWRLRLCIFLIWLYSTLVFLPSFFHWGKPG
YHGDVFQWCAESWHTDSYFTLFIVMMLYAPAALIVCFTYFNIFRICQQHTKDISE
RQARFSSQSGETGEVQACPDKRYAMVLFRITSVFYILWLPYIIYFLLESSTGHSNR
FASFLTTWLAISNSFCNCVIYSLSNSVFQRGLKRLSGAMCTSCASQTTANDPYTVR
SKGPLNGCHI

The sequence is identical with that disclosed in WO96/39441 except that the codon at position 257 encodes isoleucine.

SEQ ID NO 2 (Polynucleotide) The sequence is identical with the coding region of the polynucleotide sequence disclosed in WO96/39441.

### EXAMPLE

The transfection of a mammalian expression vector CDN containing orphan receptor HIBEF51 into the stable human embryonic kidney (HEK293) cell line resulted in at least a ten fold increase in the basal levels of cAMP accumulation. Transfection of the same expression vector not containing HIBEF51 did not result in elevated basal rates of cAMP accumulation.

1. A method for identifying compounds which inhibit the HIBEF51 receptor activity which comprises:
(a) contacting a candidate compound with cells which express the polypeptide of SEQ ID NO: 1, or with cell membranes prepared from said cells, in the absence of any ligand; and
(b) observing the inhibition of adenylyl cyclase activity.

2. A method as claimed in claim 1 in which the HIBEF51 receptor is expressed within the outer membrane of a host cell.

3. A method as claimed in claim 1 or 2 in which the cell line is mammalian HEK293, COS or CHO or frog melanophore.

4. A method as claimed in any preceding claim in which the observation (b) is of the reduction of the production and/or accumulation of cAMP.

5. A compound identified as an inhibitor of the HIBEF51 receptor activity for use in therapy.

6. A method of treating an abnormal condition related to an excess of HIBEF51 receptor activity which comprises administering to a patient in need thereof an inhibitor as claimed in claim 5 in an amount effective to inhibit receptor activity, and thereby alleviating the abnormal condition.

7. A pharmaceutical composition comprising a compound identified by the method defined in any one claims 1 to 4 and a pharmaceutically acceptable excipient or carrier.
